(19) United States
(12) Patent Application Publication
(10) Pub. No.: US 2026/0325824 A1
(43) Pub. Date: Oct. 22, 2026

(54) CIRCULATORY SUPPORT DEVICES, SYSTEMS, AND METHODS

(71) Applicant: BOSTON SCIENTIFIC SCIMED, INC., MAPLE GROVE, MN (US)

(72) Inventors: Cheng Chen, Minneapolis, MN (US); Corydon Carlson, Stillwater, MN (US)

(73) Assignee: BOSTON SCIENTIFIC SCIMED, INC., MAPLE GROVE, MN (US)

(21) Appl. No.: 19/648,230

(22) Filed: Apr. 15, 2026

Related U.S. Application Data
(60) Provisional application No. 63/789,667, filed on Apr. 16, 2025.

(51) Int. Cl. A61M 60/508; A61M 60/13; A61M 60/174; A61M 60/216; A61M 60/419

(52) U.S. Cl. CPC A61M 60/508; A61M 60/13; A61M 60/174; A61M 60/216; A61M 60/419; A61M 2205/103; A61M 2205/3327; A61M 2205/3334; A61M 2205/3365; A61M 2205/502; A61M 2210/125; A61M 2230/30

(57) ABSTRACT

A percutaneous circulatory support device may include an impeller and a motor configured to rotate the impeller to cause blood flow. The device may include a pressure sensor configured to measure vascular pressure and a controller operably coupled to both the motor and pressure sensor. The controller may determine a working voltage applied to the motor and a working speed of the motor. Using these parameters, the controller may calculate a transvalvular pressure and/or a blood flow rate based on the working voltage and working speed. The controller may then determine a left ventricular pressure based on the measured vascular pressure and the calculated transvalvular pressure.

### CROSS-REFERENCE TO RELATED APPLICATION

This application claims the benefit of U.S. patent application Ser. No. 63/789,667, filed Apr. 16, 2025, entitled “CIRCULATORY SUPPORT DEVICES, SYSTEMS, AND METHODS”, which is incorporated by reference herein in its entirety.

### TECHNICAL FIELD

The present disclosure pertains to mechanical circulatory support devices. More specifically, the present disclosure relates to the operation of percutaneous ventricular assist devices (PVADs).

### BACKGROUND

A wide variety of intracorporeal and extracorporeal medical devices and systems have been developed for medical use, for example, in cardiac procedures and/or for cardiac treatments. Some of these devices and systems include guidewires, catheters, catheter systems, pump devices, cardiac assist devices, and the like. These devices and systems are manufactured by any one of a variety of different manufacturing methods and may be used according to any one of a variety of methods. Of the known medical devices, systems, and methods, each has certain advantages and disadvantages. There is an ongoing need to provide alternative medical devices and systems as well as alternative methods for manufacturing and using medical devices and systems.

### BRIEF SUMMARY

This disclosure provides design, material, manufacturing method, and use alternatives for medical devices, including ventricular assist devices.

In an example, a percutaneous circulatory support device may include an impeller, a motor configured to rotate the impeller to cause blood flow through the percutaneous circulatory support device, a pressure sensor configured to measure a vascular pressure, and a controller operably coupled to the motor and the pressure sensor. The controller may be configured to determine a working voltage applied to the motor and determine a working speed of the motor. The controller may further be configured to calculate at least one of: a transvalvular pressure based on the working voltage and the working speed; or a blood flow rate through the percutaneous circulatory support device based on the working voltage and the working speed.

Alternatively, or additionally to any of the examples above, in another example, the controller is configured to determine a left ventricular pressure based on the measured vascular pressure and calculated transvalvular pressure.

Alternatively, or additionally to any of the examples above, in another example, the controller is configured to calculate the transvalvular pressure using a polynomial model of the working voltage and the working speed.

Alternatively, or additionally to any of the examples above, in another example, the polynomial model is a fifth-order polynomial model.

Alternatively, or additionally to any of the examples above, in another example, the controller is configured to perform auto-time alignment between pressure measurements and motor parameters.

Alternatively, or additionally to any of the examples above, in another example, the controller is configured to calculate the blood flow rate using a polynomial model of the working voltage and the working speed.

Alternatively, or additionally to any of the examples above, in another example, the polynomial model is a fifth-order polynomial model.

Alternatively, or additionally to any of the examples above, in another example, the controller is configured to display the determined left ventricular pressure and/or blood flow rate on a display screen of the percutaneous circulatory support device.

Alternatively, or additionally to any of the examples above, in another example, the controller is configured to determine the working voltage of the motor independent of the working speed of the motor.

In an example, a system for determining cardiac parameters includes a percutaneous blood pump including an impeller and a motor, a pressure sensor, and a display. A controller is operably coupled to the motor, the pressure sensor, and the display. The controller is configured to determine a working voltage applied to the motor and determine a working speed of the motor independent of the working voltage. The controller is further configured to calculate at least one of: a transvalvular pressure based using a first polynomial model of the working voltage and the working speed; or a blood flow rate through the percutaneous circulatory support device based using a second polynomial model of the working voltage and the working speed.

Alternatively, or additionally to any of the examples above, in another example, the controller is configured to determine a left ventricular pressure based on the measured vascular pressure and the calculated transvalvular pressure.

Alternatively, or additionally to any of the examples above, in another example, the first polynomial model is a fifth-order polynomial with cross-terms between the working voltage and the working speed.

Alternatively, or additionally to any of the examples above, in another example, the controller is configured to display the determined left ventricular pressure and/or the blood flow rate on the display.

Alternatively, or additionally to any of the examples above, in another example, the second polynomial model is a fifth-order polynomial with cross-terms between the working voltage and the working speed.

Alternatively, or additionally to any of the examples above, in another example, the working speed is measured based on a frequency of voltage switching signals generated as the motor rotates.

In an example, a method of operating a percutaneous circulatory support device includes positioning a blood pump within a patient's vasculature with the blood pump operably coupled to a controller. The method further includes collecting, at the controller, a working voltage of a motor operating the blood pump and collecting, at the controller, a working speed of the motor. The method further includes measuring an aortic pressure. The method further includes calculating, at the controller, at least one of: a transvalvular pressure based on the working voltage and the working speed; or a blood flow rate through the percutaneous circulatory support device based on the working voltage and the working speed.

Alternatively, or additionally to any of the examples above, in another example, the method further includes determining, at the controller, a left ventricular pressure based on the measured aortic pressure and the calculated transvalvular pressure.

Alternatively, or additionally to any of the examples above, in another example, calculating the transvalvular pressure includes using a first polynomial model of the working voltage and the working speed and/or calculating the blood flow rate includes using a second polynomial model of the working voltage and the working speed.

Alternatively, or additionally to any of the examples above, in another example, the first polynomial model is a fifth-order polynomial model and/or the second polynomial model is a fifth-order polynomial model.

Alternatively, or additionally to any of the examples above, in another example, the method further includes displaying the determined left ventricular pressure and/or the blood flow rate on a display.

In an example, a percutaneous circulatory support device may include an impeller, a motor configured to rotate the impeller to cause blood flow through the percutaneous circulatory support device, a pressure sensor configured to measure a vascular pressure, and a controller operably coupled to the motor and the pressure sensor, where the controller may be configured to determine a working voltage applied to the motor, determine a working speed of the motor, calculate a transvalvular pressure based on the working voltage and the working speed, and determine a left ventricular pressure based on the measured vascular pressure and calculated transvalvular pressure.

Alternatively, or additionally to any of the examples above, in another example, the controller may be configured to calculate the transvalvular pressure using a first polynomial model of the working voltage and the working speed.

Alternatively, or additionally to any of the examples above, in another example, the first polynomial model may be a fifth-order polynomial model.

Alternatively, or additionally to any of the examples above, in another example, the controller may be configured to perform auto-time alignment between pressure measurements and motor parameters.

Alternatively, or additionally to any of the examples above, in another example, the controller may be further configured to calculate a flow rate of fluid through the percutaneous circulatory support device based on the working voltage and working speed.

Alternatively, or additionally to any of the examples above, in another example, the controller may be configured to calculate the flow rate using a second polynomial model of the working voltage and the working speed.

Alternatively, or additionally to any of the examples above, in another example, the second polynomial model may be a fifth-order polynomial model.

Alternatively, or additionally to any of the examples above, in another example, the controller may be configured to display the determined flow rate on a display screen of the percutaneous circulatory support device.

Alternatively, or additionally to any of the examples above, in another example, the controller may be configured to display the determined left ventricular pressure on a display screen of the percutaneous circulatory support device.

Alternatively, or additionally to any of the examples above, in another example, the controller may be configured to display the determined flow rate of fluid through the percutaneous circulatory support device on a display screen of the percutaneous circulatory support device.

Alternatively, or additionally to any of the examples above, in another example, the controller may be configured to determine the working voltage of the motor independent of the working speed of the motor.

Alternatively, or additionally to any of the examples above, in another example, the pressure sensor may be configured to measure aortic pressure.

Alternatively, or additionally to any of the examples above, in another example, the controller may be configured to calculate the transvalvular pressure in real-time during operation of the percutaneous circulatory support device.

Alternatively, or additionally to any of the examples above, in another example, the controller may be configured to continuously monitor the working voltage and working speed during operation of the percutaneous circulatory support device.

Alternatively, or additionally to any of the examples above, in another example, the controller may be configured to display the determined left ventricular pressure on a display screen of the percutaneous circulatory support device.

In an example, a percutaneous circulatory support device may include an impeller, a motor configured to rotate the impeller to cause blood flow through the percutaneous circulatory support device, a pressure sensor configured to measure a vascular pressure, and a controller operably coupled to the motor and the pressure sensor, where the controller may be configured to determine a working voltage applied to the motor, determine a working speed of the motor, calculate a transvalvular pressure based on the working voltage and the working speed, and determine a left ventricular pressure based on the measured vascular pressure and calculated transvalvular pressure.

Alternatively, or additionally to any of the examples above, in another example, the controller may be configured to calculate the transvalvular pressure using a polynomial model of the working voltage and the working speed.

Alternatively, or additionally to any of the examples above, in another example, the polynomial model may be a fifth-order polynomial model.

Alternatively, or additionally to any of the examples above, in another example, the controller may be configured to perform auto-time alignment between pressure measurements and motor parameters.

Alternatively, or additionally to any of the examples above, in another example, the controller may be further configured to calculate a flow rate of fluid through the percutaneous circulatory support device based on the working voltage and working speed.

Alternatively, or additionally to any of the examples above, in another example, the controller may be configured to calculate the flow rate using a polynomial model of the working voltage and the working speed.

Alternatively, or additionally to any of the examples above, in another example, the polynomial model may be a fifth-order polynomial model.

Alternatively, or additionally to any of the examples above, in another example, the controller may be configured to display the determined left ventricular pressure on a display screen of the percutaneous circulatory support device.

Alternatively, or additionally to any of the examples above, in another example, the controller may be configured to determine the working voltage of the motor independent of the working speed of the motor.

In an example, a system for determining cardiac parameters may include a percutaneous blood pump including an impeller and motor, a pressure sensor, a display, and a controller operably coupled to the motor, pressure sensor, and display, where the controller may be configured to determine a working voltage applied to the motor, determine a working speed of the motor independent of the working voltage, calculate a transvalvular pressure based using a polynomial model of the working voltage and the working speed, and determine a left ventricular pressure based on the measured vascular pressure and the calculated transvalvular pressure.

Alternatively, or additionally to any of the examples above, in another example, the polynomial model may be a fifth-order polynomial with cross-terms between the working voltage and the working speed.

Alternatively, or additionally to any of the examples above, in another example, the controller may be configured to display the determined left ventricular pressure on the display.

Alternatively, or additionally to any of the examples above, in another example, the controller may be further configured to calculate a flow rate of fluid through the percutaneous blood pump based on the working voltage and working speed.

Alternatively, or additionally to any of the examples above, in another example, the controller may be configured to calculate the flow rate using a polynomial model of the working voltage and the working speed.

Alternatively, or additionally to any of the examples above, in another example, the polynomial model may be a fifth-order polynomial with cross-terms between the working voltage and the working speed.

In an example, a method of operating a percutaneous circulatory support device may include positioning a blood pump within a patient's vasculature where the blood pump may be operably coupled to a controller, collecting at the controller a working voltage of a motor operating the blood pump, collecting at the controller a working speed of the motor, measuring an aortic pressure, calculating at the controller a transvalvular pressure based on the working voltage and the working speed, and determining at the controller a left ventricular pressure based on the measured aortic pressure and the calculated transvalvular pressure.

Alternatively, or additionally to any of the examples above, in another example, the method may further include calculating at the controller a flow rate of fluid through the blood pump based on the working voltage and working speed.

Alternatively, or additionally to any of the examples above, in another example, calculating the transvalvular pressure may include using a polynomial model of the working voltage and the working speed.

Alternatively, or additionally to any of the examples above, in another example, the polynomial model may be a fifth-order polynomial model.

Alternatively, or additionally to any of the examples above, in another example, the method may further include displaying the determined left ventricular pressure.

The above summary of some embodiments is not intended to describe each disclosed embodiment or every implementation of the present disclosure. The Figures, and Detailed Description, which follow, more particularly exemplify some of these embodiments.

### BRIEF DESCRIPTION OF THE DRAWINGS

The disclosure may be more completely understood in consideration of the following detailed description in connection with the accompanying drawings, in which:

FIG. 1 is a schematic partial cross-section of anatomy and a schematic side view of an illustrative percutaneous ventricular assist device (PVAD) within the anatomy;

FIG. 2 is a schematic cross-sectional view of an illustrative PVAD;

FIG. 3 is a schematic detailed view of the illustrative PVAD depicted in FIG. 2, taken within line 3-3;

FIG. 4 is a schematic diagram of an illustrative circulatory support system;

FIG. 5 is a schematic diagram of an illustrative computing device or controller and user interface;

FIG. 6 is a flow chart of an illustrative method for determining left ventricle pressure; and

FIG. 7 is a flow chart of an illustrative method for determining a flow rate through a PVAD.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the term “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar structures in different drawings are numbered the same. The drawings, which are not necessarily to scale, depict illustrative embodiments and are not intended to limit the scope of the disclosure. Additionally, it should be noted that in any given figure, some features may not be shown, or may be shown schematically, for clarity and/or simplicity. Additional details regarding some components and/or method steps may be illustrated in other figures in greater detail. The devices and/or methods disclosed herein may provide a number of desirable features and benefits as described in more detail below.

A variety of circulatory assist devices are known for assisting or replacing a pumping function of a heart in a patient with severe heart failure and/or other cardiac conditions. Circulatory assist devices may be configured to treat patients with cardiogenic shock, myocardial infarction, acutely decompensated heart failure, and/or other heart related conditions. Additionally, or alternatively, circulatory assist devices may support a patient during percutaneous coronary interventions and/or other procedures.

Example cardiac circulatory assist devices include, but are not limited to, ventricular assist devices (VADs), total artificial hearts, intra-aortic balloon pumps (IABP), and extracorporeal membrane oxygenation (ECMO). Example VADs include left ventricular assist devices (LVADs), right ventricular assist devices (RVADs), and biventricular assist devices (BiVADs). A further illustrative VAD is a percutaneous ventricular assist device (PVAD), which may be inserted into a ventricle (e.g., a left ventricle or a right ventricle) of a heart of a patient via delivery through a femoral artery or vein and/or other suitable vasculature to the ventricle. A PVAD may be placed at a desired location of anatomy of a patient via percutaneous access and delivery, which may enable the PVAD to be used in emergency medicine, a cath lab, and/or other surgical and/or non-surgical settings.

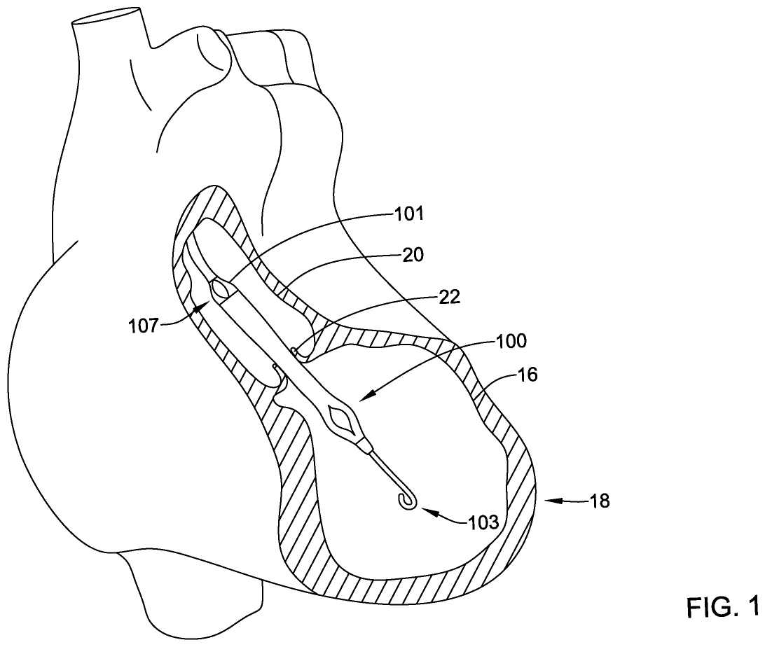

FIG. 1 depicts an illustrative positioning of a blood pump 100 (e.g., a percutaneous circulatory support device, such as a PVAD in an LVAD configuration, etc.) in the anatomy of a patient. In FIG. 1, the blood pump 100 is positioned with a distal end 103 located in a left ventricle 16 of a heart 18 and a proximal end 107 located in an aorta 20, such that the blood pump 100 extends across an aortic valve 22 between the left ventricle 16 and the aorta 20. With the blood pump 100 extending from the left ventricle 16 to the aorta 20, the blood pump 100 may be configured to pump blood from the left ventricle 16 into the aorta 20 to assist blood flow circulation. Other suitable positions of the blood pump relative to the anatomy are contemplated and include, but are not limited to, the distal end 103 of the blood pump being positioned in a right ventricle of the heart 18 with a proximal end being positioned in a pulmonary artery.

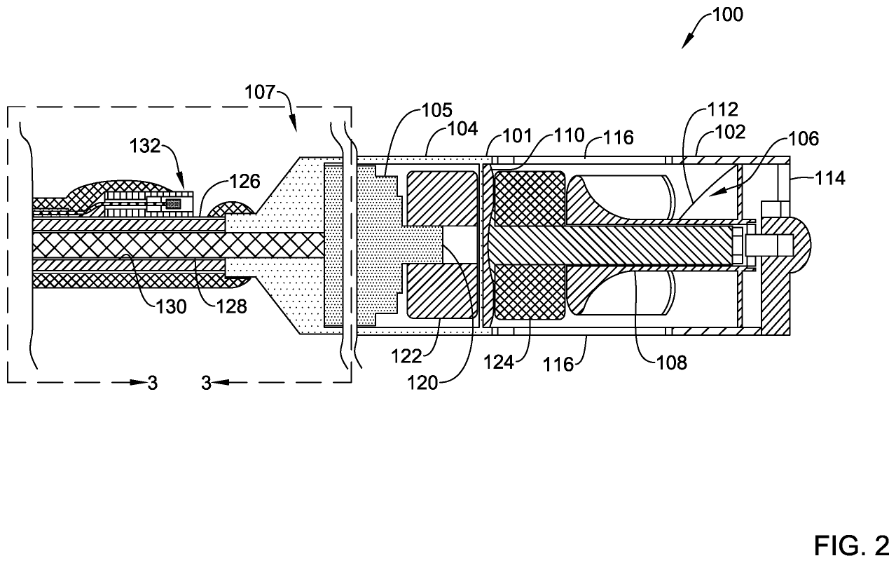

FIG. 2 depicts a schematic cross-sectional view of an illustrative configuration of the blood pump 100. In some cases, the blood pump 100 may form part of a percutaneous circulatory or circulatory support system, together with a guidewire, an introducer sheath, a controller, a user interface, one or more sensors, and/or other suitable components. The blood pump 100 may include a housing 101 having an impeller housing 102 and a motor housing 104. The impeller housing 102 and the motor housing 104 may be formed as single monolithic piece, but this is not required. In some cases, the impeller housing 102 and the motor housing 104 may be separate components configured to be removably or permanently coupled. In some configurations, the blood pump 100 may lack a motor housing 104 separate from the impeller housing 102, and the impeller housing 102 may be coupled directly to a motor 105, or the motor housing 104 may be integrally constructed with the motor 105.

The impeller housing 102 may house an impeller assembly 106 and a driven magnet 124, which may be part of or separate from the impeller assembly 106. The impeller assembly 106 may include an impeller shaft 108 that is rotatably supported by at least one bearing, such as a bearing 110 and/or other suitable bearings. The impeller assembly 106 may further include an impeller 112 that rotates relative to the impeller housing 102 to drive blood through the blood pump 100. In some configurations and, for example as illustrated, the impeller shaft 108 and the impeller 112 may be separate components, and in other configurations the impeller shaft 108 and the impeller 112 may be integrated. The impeller assembly 106, as a whole, may be considered a driven component and/or the rotating components of the impeller assembly 106 (e.g., the impeller shaft 108 and/or the impeller 112) may individually or in combination be driven components.

The impeller 112 may be configured within the impeller housing 102 such that as the impeller 112 rotates, blood flows from a blood inlet 114 formed on or at the impeller housing 102, through the impeller housing 102, and out of a blood outlet 116 formed on or at the impeller housing 102. In some configurations, the impeller housing 102 may couple to or include a distally extending cannula (not shown), and the cannula may receive and deliver blood to the inlet 114 (e.g., from the left ventricle 16 of the heart 18 and/or from other suitable locations).

The inlet 114 and the outlet 116 may each have any suitable number of apertures configured to facilitate receiving blood at the blood pump 100 and outputting blood from the blood pump 100, respectively. In some examples, the inlet 114 and/or the outlet 116 may each include multiple apertures and in other examples, one or both of the inlet 114 and the outlet 116 may each include a single aperture.

The inlet 114 and the outlet 116 may each be formed at any suitable location along the impeller housing 102 or other suitable location along the blood pump 100. In some examples, and as depicted in FIG. 2, the inlet 114 may be formed on an end portion (e.g., a distal end portion) of the impeller housing 102 and the outlet 116 may be formed on a side portion (e.g., proximal of the location of the inlet 114) of the impeller housing 102. Other suitable positioning configurations of the inlet 114 and/or the outlet 116 on the impeller housing 102 are contemplated.

The motor housing 104 may house the motor 105, along with other suitable components. In some examples, and as depicted in FIG. 2, the motor housing 104 may house at least the motor 105, a drive shaft 120, and a driving magnet 122. The motor 105 may be any suitable type of motor. In one example, the motor 105 may be a brushless direct current (DC) motor (BLDC), but other suitable motor types are contemplated.

In operation, the motor 105 may be configured to rotatably drive the impeller 112 relative to the impeller housing 102. In some example configurations, the motor 105 may rotate a drive shaft 120, which is coupled to a driving magnet 122. Rotation of the driving magnet 122 may cause rotation of the driven magnet 124, which is part of or connected to the impeller assembly 106 and rotates together with the impeller assembly 106. That is, when the impeller shaft 108 is included in the impeller assembly 106, the impeller shaft 108 and the impeller 112 are configured to rotate with the driven magnet 124. Additionally, or alternatively, the motor 105 may couple to the impeller assembly 106 via other components.

A controller (not shown in FIG. 2) may be operably coupled to the motor 105 and configured to control the motor 105 via one or more command signals sent from the controller to the motor 105. The controller may be disposed within the motor housing 104 and/or the controller may be disposed outside of the motor housing 104 (e.g., in a housing of the blood pump 100 independent from the motor housing 104, exterior of the patient, etc.). In some embodiments, the controller may include multiple components, one or more of which may be disposed within and/or separately from the motor housing 104.

The motor housing 104 may couple to a catheter 126 at a location of the motor housing 104 opposite the impeller housing 102. The catheter 126 may couple to the motor housing 104 in various manners, such as laser welding, soldering, or the like. The catheter 126 may extend proximally away from the motor housing 104.

The catheter 126 may include one or more lumens for receiving one or more components of a circulatory support system including the blood pump 100. In some cases, the catheter 126 may be configured to carry a motor cable 128 (e.g., one or more cables configured to facilitate operation of the motor 105) within a main lumen 130, and the motor cable 128 may operably couple the motor 105 to the controller (not shown) and/or an external power source (not shown).

The catheter 126 may carry a sensor assembly 132 for measuring pressure within the vasculature of a patient, for example, within the aorta or pulmonary artery. The sensor assembly 132 may be positioned, relative to the other components of the blood pump 100, in a location for obtaining highly accurate pressure data. For example, the proximal position of the sensor assembly 132 relative to the motor housing 104 and the motor 105 may reduce and/or eliminate the motor speed-related or dynamic pressure-related sensing inaccuracies. Such inaccuracies are typical of other percutaneous circulatory support devices that employ pressure sensors located more distally relative to the motor or impeller assembly, for example, devices that employ pressure sensors located near the outlet 116.

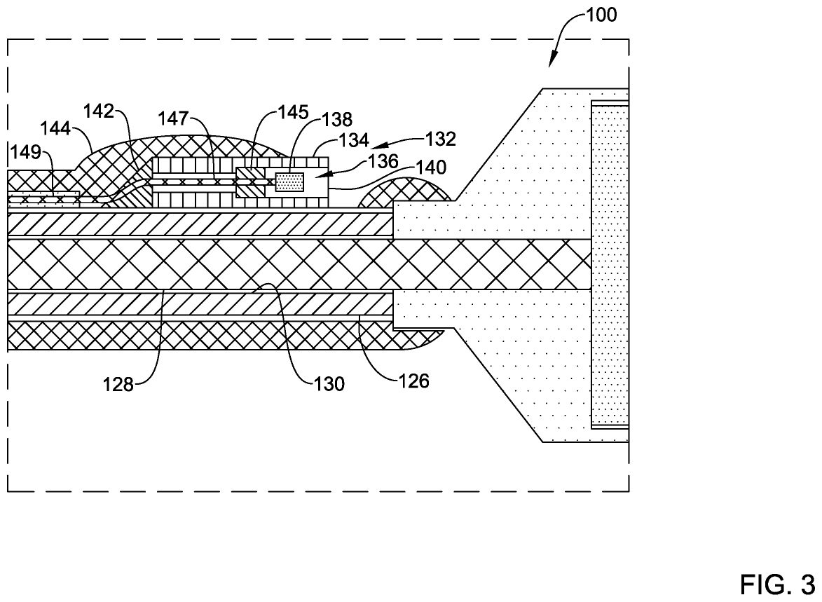

FIG. 3 depicts a schematic detailed view of an interior of line 3-3 in FIG. 2. As depicted in FIG. 3, the sensor assembly 132 may include a sensor housing 134 having an internal chamber 136. In some examples, the internal chamber 136 may have a counterbore-shape, but other suitable shapes and/or configurations of the internal chamber 136 are contemplated. A pressure sensor 138, such as an optical pressure sensor (e.g., an optical pressure sensor using one or more fiber optics and/or other suitable optical pressure sensor), an electrical pressure sensor, and/or other suitable pressure sensors, may be disposed within the internal chamber 136 and configured to sense a pressure in the aorta 20 when the blood pump 100 extends into the left ventricle 16 of the heart 18. The sensor housing 134 may protect the pressure sensor 138 during deployment of the blood pump 100. The sensor housing 134 may also include a distally facing aperture 140 formed in or coupled to the internal chamber 136. The aperture 140 may permit blood to enter the internal chamber 136, and the aperture 140 thereby permits the pressure sensor 138 to sense the pressure of the blood proximate the internal chamber 136.

The sensor housing 134 may take various forms. For example, the sensor housing 134 may be a tube or ferrule manufactured from, for example, one or more metals, one or more plastics, composites, and/or other suitable materials. The sensor housing 134 may be coupled to the catheter 126 via one or more weldments (not shown), one or more adhesives 142, and/or an outer jacket 144 surrounding at least a portion of the sensor housing 134 and the catheter 126. The sensor housing 134 may also include a sensor mount 145 within the internal chamber 136. The sensor mount 145 may facilitate supporting the pressure sensor 138 spaced from the walls of the sensor housing 134 (e.g., the sensor mount 145 may center the pressure sensor 138 within the internal chamber 136), which in turn facilitates high-accuracy pressure sensing. Other suitable configurations of the sensor housing 134 are contemplated.

The sensor assembly 132 may include a sensor cable 147 coupled to the pressure sensor 138. The sensor cable 147 may operably couple the pressure sensor 138 to the controller (not shown). As illustrated, the sensor cable 147 may extend through the sensor mount 145 and support the pressure sensor 138 apart from the walls of the sensor housing 134. The sensor cable 147 may extend proximally, through the adhesive 142, and through a cable lumen 149 of or coupled to the catheter 126. In some examples, the cable lumen 149 may be coupled to the catheter 126 via one or more weldments (not shown), an adhesive (not shown), and/or the outer jacket 144. In other examples, the cable lumen 149 may be omitted, and the sensor cable 147 may extend through the main lumen 130 of the catheter 126 or lie directly under the outer jacket 144. Example suitable sensor assemblies 132 are disclosed in U.S. Patent Application Publication No. 2023/0149699 A1 , filed on Nov. 16, 2022, and titled PERCUTANEOUS CIRCULATORY SUPPORT DEVICE INCLUDING PROXIMAL PRESSURE SENSOR, which is hereby incorporated by reference in its entirety.

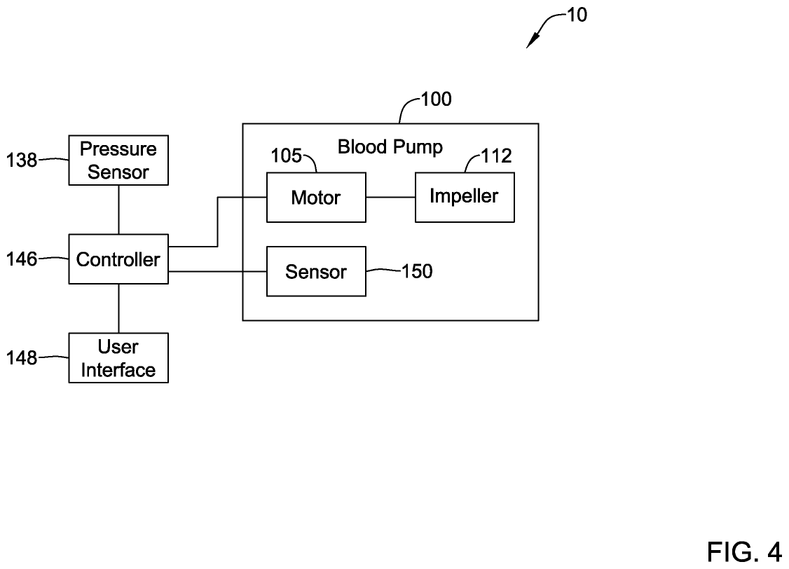

FIG. 4 depicts a schematic diagram of an illustrative circulatory support system 10. Among other additional and/or alternative components, the circulatory support system 10 may include the blood pump 100, the pressure sensor 138, a controller 146, and a user interface 148. As discussed, the blood pump 100 may include a motor 105 in communication with the controller 146 and the impeller 112 in communication with the motor 105.

In some examples, the blood pump 100 may include or be coupled to one or more sensors 150 (e.g., one or more position sensors and/or other suitable speed sensors) configured to sense a speed of the motor 105. When one or more of the sensors 150 are included, the one or more sensors 150 may be coupled to the controller 146 via one or more cables extending through and/or along the catheter 126. In some cases, the speed or position of the motor 105 may be sensed directly from the electrical signals used to drive the motor 105 by the controller 146. In such cases, the motor may be an implicit sensor used with or obviating the need for an explicit sensor 150.

The one or more sensors 150 may be any suitable type(s) of sensor for sensing a speed of a motor. Example suitable type(s) of sensors 150 include, but are not limited to, position sensors, Hall effect sensors, magnetic inductive sensors, optical encoders, eddy current sensors, doppler effect sensors, tachometers, and/or other suitable types of sensors.

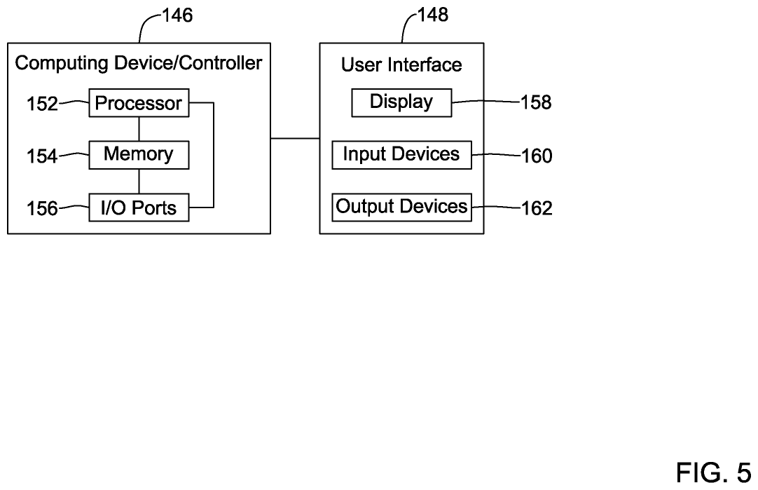

FIG. 5 depicts a schematic diagram of an illustrative configuration of the controller 146 (e.g., computing device) and the user interface 148 of the circulatory support system 10. The controller 146 may be and/or may include any suitable computing device configured to process data of or for the circulatory support system 10 (e.g., of or from the motor 105, the pressure sensor 138, the sensor 150, patient test results, user inputs, patient monitors, or the like). In some cases, one or more components of the circulatory support system 10 may be incorporated into the controller 146 and/or the user interface 148. Further, one or more components of the circulatory support system 10 may incorporate one or more computing devices similar to or having components similar to the controller 146 and/or the user interface 148.

The controller 146 may be configured to facilitate operation of the circulatory support system 10. The controller 146, in some cases, may be configured to control operation of the motor 105, the pressure sensor 138, the user interface 148, and/or the sensor 150 by establishing and/or outputting control signals to components of the motor 105, the pressure sensor 138, the user interface 148, and/or the sensor 150 to control and/or monitor operation of these units and devices.

The controller 146 may communicate with a remote server or other suitable computing device. When the controller 146, or at least a part of the controller 146, is a component separate from a structure of the motor 105, the pressure sensor 138, the user interface 148, and/or the sensor 150, the controller 146 may communicate with electronic components of the circulatory support system 10 over one or more wired or wireless connections or networks (e.g., LANs and/or WANs).

The controller 146 may be, may include, or may be included in one or more Field Programmable Gate Arrays (FPGAs), one or more Programmable Logic Devices (PLDs), one or more Complex PLDs (CPLDs), one or more custom Application Specific Integrated Circuits (ASICs), one or more dedicated processors (e.g., microprocessors), one or more Central Processing Units (CPUs), software, hardware, firmware, or any combination of these and/or other components. Although the controller 146 may be referred to herein in the singular, the controller 146 may be implemented in multiple instances, distributed across multiple computing devices, instantiated within multiple virtual machines, and/or the like.

The illustrative controller 146 may include, among other suitable components, one or more processors 152, memory 154, and/or one or more input/output (I/O) units 156. Examples of other suitable components of the controller 146 that are not specifically depicted in FIG. 2 may include, but are not limited to, communication components, a touch screen, selectable buttons, a housing, and/or other suitable components of a controller. As discussed above, one or more components of the controller 146 may be separate from the components of the circulatory support system 10 and/or incorporated into the components of the circulatory support system 10.

The controller 146 may include and/or be in communication with a variety of sub-controllers. Example sub-controllers that may be included in or in communication with the controller 146 may include, but is not limited to, a motor sub-controller, a flow rate sub-controller, a pressure sub-controller, a motor torque sub-controller, a motor mechanical loss sub-controller, a stall pressure sub-controller, a pressure loss sub-controller, and/or other suitable sub-controllers.

The processor 152 of the controller 146 may include a single processor or more than one processor working individually or with one another. The processor 152 may be configured to receive and execute instructions, including instructions that may be loaded into the memory 154 and/or other suitable memory. Example components of the processor 152 may include, but are not limited to, central processing units, microprocessors, microcontrollers, multi-core processors, graphical processing units, digital signal processors, application specific integrated circuits (ASICs), artificial intelligence accelerators, field programmable gate arrays (FPGAs), discrete circuitry, and/or other suitable types of data processing devices.

The memory 154 of the controller 146 may include a single memory component or more than one memory component each working individually or with one another. Example types of memory 154 may include random access memory (RAM), EEPROM, flash, suitable volatile storage devices, suitable non-volatile storage devices, persistent memory (e.g., read only memory (ROM), hard drive, flash memory, optical disc memory, and/or other suitable persistent memory) and/or other suitable types of memory. The memory 154 may be or may include a non-transitory computer readable medium. The memory 154 may include instructions stored in transitory and/or non-transitory state on a computer readable medium that may be executable by the processor 152 to cause the processor to perform one or more of the methods and/or techniques described herein.

The I/O units 156 of the controller 146 may include a single I/O component or more than one I/O component each working individually or with one another. Example I/O units 156 may be or may include any suitable types of communication hardware and/or software including, but not limited to, communication ports configured to communicate with electronic components of the circulatory support system 10 and/or with other suitable computing devices or systems. Example types of I/O units 156 may include, but are not limited to, wired communication components (e.g., HDMI components, Ethernet components, VGA components, serial communication components, parallel communication components, component video ports, S-video components, composite audio/video components, DVI components, USB components, optical communication components, and/or other suitable wired communication components), wireless communication components (e.g., radio frequency (RF) components, Low-Energy BLUETOOTH protocol components, BLUETOOTH protocol components, Near-Field Communication (NFC) protocol components, WI-FI protocol components, optical communication components, ZIGBEE protocol components, and/or other suitable wireless communication components), and/or other suitable I/O units 156.

The user interface 148 may be configured to communicate with the controller 146 via one or more wired or wireless connections. The user interface 148 may include one or more display devices 158, one or more input devices 160, one or more output devices 162, and/or one or more other suitable features.

The display device 158 may be any suitable display. Example suitable displays include, but are not limited to, touch screen displays, non-touch screen displays, liquid crystal display (LCD) screens, light emitting diode (LED) displays, head mounted displays, virtual reality displays, augmented reality displays, and/or other suitable display types.

The input device(s) 160 may be and/or may include any suitable components and/or features for receiving user input via the user interface. Example input device(s) 160 include, but are not limited to, touch screens, keypads, mice, touch pads, microphones, selectable buttons, selectable knobs, optical inputs, cameras, gesture sensors, eye trackers, voice recognition controls (e.g., microphones coupled to appropriate natural language processing components), and/or other suitable input devices.

The output device(s) 162 may be and/or may include any suitable components and/or features for providing information and/or data to users and/or other computing components. Example output device(s) 162 include, but are not limited to, displays, speakers, vibration systems, tactile feedback systems, optical outputs, cables, lights, and/or other suitable output devices.

Various parameters (e.g., pressure, flow, etc.) of, through, or proximate the blood pump 100 may be operationally and/or clinically relevant performance parameters of or relative to the blood pump 100 positioned within a patient. For example, clinicians may be interested in having ventricular pressure data (i.e., pressure within the ventricle), vasculature pressure data (e.g., aortic pressure data, pulmonary artery pressure data, etc.), differential pressure data across the blood pump 100 (e.g., between the ventricle and aorta, etc.) such as transvalvular pressure, flow data related to blood flowing through the blood pump 100, impeller speed data, motor speed data and/or other data related to operation of the blood pump 100 for making decisions concerning additional or alternative therapies, assessing a condition of the patient, assessing a condition of the blood pump, assessing operation of the blood pump, assessing effectiveness of the blood pump, controlling operation of the blood pump, etc. Further, pressure and flow data related to and/or for blood flowing through the blood pump 100, other blood pump data related to operation of the blood pump 100, and/or calculations based on data related to blood flowing through the blood pump 100 may be utilized to control operation of the blood pump 100 (e.g., adjust command signals, etc.) automatically by the controller 146 and/or in response to user input.

Management of patients with mechanical circulatory support (MCS) devices (e.g., PVADs, such as the blood pump 100 and/or other suitable blood pumps) positioned within their heart may depend on one or more variables and thus, it may not always be clear to a user (e.g., physicians, clinicians, etc.) which pump to use to treat the patient (e.g., a pump with lower flow rate capabilities versus a pump with higher flow rate capabilities). As higher flow rate pumps may be more likely to cause adverse effects (e.g., hemolysis, etc.) on a patient than lower flow rate pumps, users may tend to initially deliver a lower flow rate pump to a patient with an understanding that a higher flow rate pump may be needed in the future. Additionally, or alternatively, needs of a patient may change during treatment and a higher flow rate blood pump or a lower flow rate blood pump may be required or preferred. Further, the capabilities of a blood pump may reduce over time, such that a new, higher flow rate pump is required to meet patient needs. When the needs of a patient exceed capabilities of a blood pump currently being used to treat the patient, a blood pump transition to a higher flow rate blood pump or multiple blood pumps is required (e.g., a device escalation). Further, a blood pump transition to a lower flow rate pump is contemplated, for example, when the needs of the patient for circulatory support are reduced (e.g., a device de-escalation or demotion).

When the blood pump 100 is positioned in an LVAD configuration, it may be desirable to know or estimate the pressure within the left ventricle 16. While the present disclosure is described with respect to the blood pump 100 positioned with the distal end 103 located in the left ventricle 16 and the proximal end 107 located in the aorta 20, the systems and methods described herein may be used to determine flow rate and/or pressure when the blood pump 100 is positioned in other configurations (e.g., RVAD) or other parts of the anatomy. It is contemplated that the pressure of the left ventricle 16 (i.e., left ventricular pressure) may be used to estimate a position of the blood pump 100, estimate or calculate cardiac power and/or output, and/or gauge heart failure. It may be desirable for the circulatory support system 10 to determine (e.g., estimate) the pressure of the left ventricle 16 and/or a flow rate through the blood pump 100 without the use of additional sensors or hardware.

Generally, experimental data may be used to approximate transvalvular pressure as an independent function of the working speed of the motor 105 and the working voltage of the motor 105. The left ventricle pressure may then be calculated based on the measured pressure in the aorta 20 (as measured at the pressure sensor 138) and the approximated or estimated transvalvular pressure. For example, the left ventricle pressure may be determined using Equation 1:

P_{LV}=P_{AO}−P_{TV} Equation 1

where P_{LV} is the pressure in the left ventricle, P_{AO} is the pressure in the aorta and P_{TV} is the transvalvular pressure. Transvalvular pressure is the pressure difference between the blood pressure on one side of a heart valve (e.g., the aortic valve) and the pressure on the other side of the heart valve. In the present example, the transvalvular pressure is the pressure difference between the blood pressure in the aorta and the pressure in the left ventricle. As will be described in more detail herein, transvalvular pressure may be estimated using Equation 2:

P_{TV}=f(voltage,speed) Equation 2

where P_{TV} is the transvalvular pressure, voltage is the working voltage of the motor 105 and speed is the working speed of the motor 105. For example, f (voltage, speed) may be a polynomial model as described with more detail with respect to Equation 4. The working voltage of the motor 105 and the working speed of the motor 105 may be determined as described herein. It is further contemplated that the flow rate of blood through the blood pump 100 may also be estimated using Equation 3:

Flow Rate=f(speed,voltage) Equation 3

where flow rate is the flow rate of blood through the blood pump 100, voltage is the working voltage of the motor 105 and speed is the working speed of the motor 105. For example, f (speed, voltage) may be a polynomial model. The working voltage of the motor 105 and the working speed of the motor 105 may be determined as described herein. While both transvalvular pressure and flow rate are both determined as functions of motor working speed and motor working voltage, Equations 2 and 3 may be different.

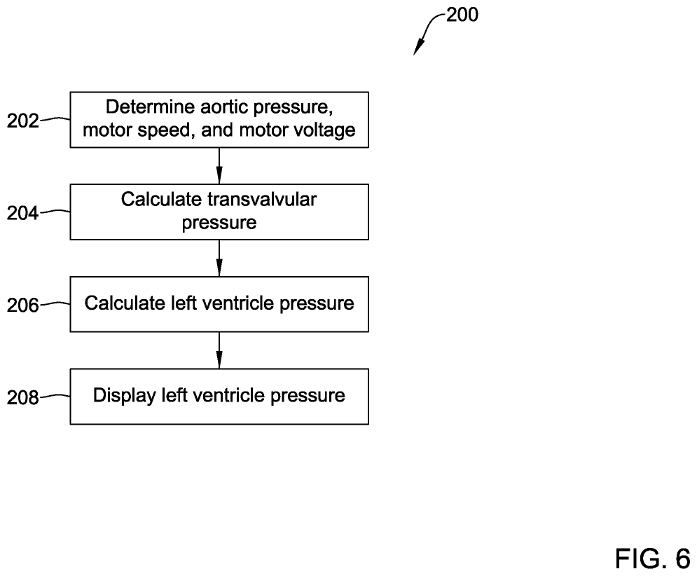

FIG. 6 is an illustrative flow chart of a method 200 for estimating the pressure in the left ventricle (i.e., estimating left ventricular pressure). Prior to performing the method 200, the circulatory support system 10 may be deployed and the blood pump 100 positioned within the patient. For example, the blood pump 100 may be positioned such that the distal end 103 is located in the left ventricle 16 and the proximal end 107 is located in the aorta 20. To begin the left ventricle pressure estimation, the controller 146 may collect and/or determine an aortic pressure from pressure data measured by the pressure sensor 138, a motor working speed, and a motor working voltage, as shown at block 202. It is contemplated that the aortic pressure, motor working speed, and motor working voltage may be captured simultaneously or substantially simultaneously. In some cases, the controller 146 may be configured to continually collect, receive, determine, and/or capture the aortic pressure, motor working speed, and motor working voltage at predetermined time intervals. In some examples the motor speed and/or the motor working voltage may be filtered or augmented using additional values such as reference values for control loops and command values for digital to analog converters. These augmentations may help to overcome delay values caused by delays in sensors and filtering to remove noise.

Motor working speed and motor working voltage may each be affected by different effects of the human body. Thus, the motor working speed may be measured independently of motor working voltage and vice versa. It is contemplated that motor working speed may be measured using voltage switches (e.g., the sequential energization/de-energization of the stator windings in the motor 105). For example, the frequency of voltage switching signals generated by the motor's 105 electronic speed controller as the motor 105 rotates may be monitored. This frequency of voltage switches may be directly correlated with the rotational speed of the motor 105. Thus, the speed, i.e., revolutions per minute (RPM), of the motor 105 can be determined by counting the number of voltage switches per unit time for a motor 105 having a known quantity of stator windings. For example, for a motor having three stator windings, the motor would experience six voltage switches per revolution. Thus, knowing the frequency of voltage switches and the quantity of stator windings of the motor, the speed of the motor can be determined. Alternatively, or additionally, the working speed of the motor 105 may be determined by monitoring the rotor position using sensors, such as, but not limited to Hall effect sensors. The working voltage of the motor 105 may be determined through direct measurements from the motor windings. The voltage may also be measured at the supply feeding the switches that control voltage to the motor windings.

The controller 146 may calculate the transvalvular pressure as a function of the working speed of the motor 105 and the working voltage of the motor 105, as shown at block 204. The calculated transvalvular pressure is an estimated pressure calculated using a polynomial model based on the working speed of the motor 105 and the working voltage of the motor 105. It is contemplated that experimental data may be used to generate a model that estimates or predicts the transvalvular pressure. This model may be stored in the memory 154 of the controller 146. As the controller 146 receives the working speed of the motor 105 and the working voltage of the motor 105 the processing unit 152 may input the determined working speed and working voltage into the model to calculate the transvalvular pressure. In some cases, the controller 146 may be configured to automatically perform time alignment between the motor parameters (e.g., working speed and working voltage) and the pressure measurements from the pressure sensor 138.

Generally, the model for determining the transvalvular pressure may be generated by measuring transvalvular pressure in a controlled environment while varying the working speed of the motor 105 and measuring the working voltage of the motor 105. This may be repeated using different devices of the same type. A best fit model for the experimental data may then be generated using statistical or machine learning techniques. In some cases, the model may be a first polynomial model that is a 5^{th}-order polynomial. However, this is not required. An illustrative 5^{th} order polynomial may take the following form identified in Equation 4:

P_{TV}=a+bS+cV+dS²+eSV+fV²+gS³+hS²V+iSV²+jV³+kS⁴+lS³V+mS²V²+nSV³+oV⁴+pS⁵+qS⁴V+rS³V²+tS²V³+uSV⁴+wV⁵ Equation 4

where P_{TV} is the transvalvular pressure, a, b, c, d, e, f, g, h i, j, k, l, m, n, o, p, q, r, t, u, and w are experimentally derived coefficients, S is the working speed of the motor, and V is the working voltage of the motor. The model may include individual terms of working voltage and working speed as well as cross-terms between the working voltage and the working speed. Cross-terms are terms that include both the working voltage and the working speed multiplied together.

The range of values for the coefficients a through w may vary significantly (by an order of 10¹⁵) depending on the units used for speed and voltage (for example, radians and millivolts versus RPM and volts). In practice values in the range of 10³ to 10⁻¹⁵ may be reasonable for higher order coefficients.

The controller 146 may then calculate the pressure of the left ventricle 16 as shown at block 206. The pressure of the left ventricle 16 may be the pressure in the aorta 20 (as measured at the pressure sensor 138) minus the calculated transvalvular pressure (e.g., using the experimentally derived model) as set forth in Equation 1. The controller 146 may be configured to display the calculated left ventricle pressure on the display 158 of the user interface 148, as shown at block 208. The controller 146 may be configured to automatically calculate and display the pressure of the left ventricle (e.g., without requiring user intervention) in real time. In some cases, the controller 146 may calculate and display the pressure of the left ventricle continuously or at pre-defined intervals.

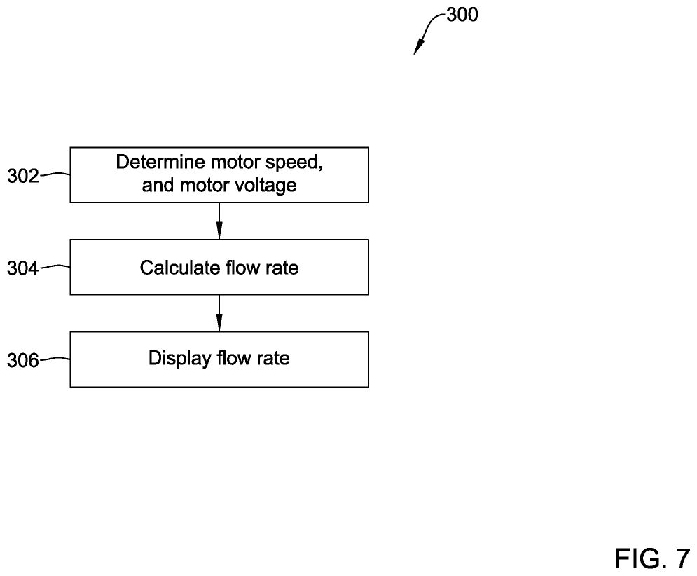

FIG. 7 is an illustrative flow chart of a method 300 for estimating the flow rate of blood through the blood pump 100. Prior to performing the method 300, the circulatory support system 10 may be deployed and the blood pump 100 positioned within the patient. For example, the blood pump 100 may be positioned such that the distal end 103 is located in the left ventricle 16 and the proximal end 107 is located in the aorta 20. To begin the flow rate estimation, the controller 146 may collect and/or determine a motor working speed and a working motor voltage, as shown at block 302. It is contemplated that the motor working speed and motor working voltage may be captured simultaneously or substantially simultaneously. In some cases, the controller 146 may be configured to continually collect, receive, determine, and/or capture the motor speed, and motor voltage at predetermined time intervals.

Motor working speed and motor working voltage may each be affected by different effects of the human body. Thus, the motor working speed may be measured independently of motor working voltage and vice versa. It is contemplated that motor working speed may be measured using voltage switches (e.g., the sequential energization/de-energization of the stator windings in the motor 105). For example, the frequency of voltage switching signals generated by the motor's 105 electronic speed controller as the motor 105 rotates may be monitored. This frequency of voltage switches may be directly correlated with the rotational speed of the motor 105. Thus, the speed, i.e., revolutions per minute (RPM), of the motor 105 can be determined by counting the number of voltage switches per unit time for a motor 105 having a known quantity of stator windings. For example, for a motor having three stator windings, the motor would experience six voltage switches per revolution. Thus, knowing the frequency of voltage switches and the quantity of stator windings of the motor, the speed of the motor can be determined. Alternatively, or additionally, the working speed of the motor 105 may be determined by monitoring the rotor position using sensors, such as, but not limited to Hall effect sensors. The working voltage of the motor 105 may be determined through direct measurements from the motor windings. The voltage may also be measured at the supply feeding the switches that control voltage to the motor windings.

The controller 146 may calculate the flow rate as a function of the working speed of the motor 105 and the working voltage of the motor 105, as shown at block 304. The calculated flow rate is an estimated flow rate calculated using a polynomial model based on the working speed of the motor 105 and the working voltage of the motor 105. It is contemplated that experimental data may be used to generate a model that estimates or predicts the flow rate. This model may be stored in the memory 154 of the controller 146. As the controller 146 receives the working speed of the motor 105 and the working voltage of the motor 105 the processing unit 152 may input the determined working speed and working voltage into the model to calculate the flow rate.

Generally, the model for determining the flow rate may be generated by measuring flow rate in a controlled environment while varying the speed of the motor 105 and measuring the voltage of the motor 105. This may be repeated using different devices of the same type. A best fit second polynomial model for the experimental data may then be generated. In some cases, the model may be a 5^{th}-order polynomial. However, this is not required. A 5^{th}-order polynomial may be similar to Equation 4 above. However, the experimentally derived coefficients for the model to determine flow rate may be different from the coefficients for the model to determine transvalvular pressure. The flow rate model may include individual terms of working voltage and working speed as well as cross-terms between the working voltage and the working speed.

The range of values for the coefficients a through w for the second polynomial model may vary significantly (by an order of 10¹⁵) depending on the units used for speed and voltage (for example, radians and millivolts versus RPM and volts). In practice values in the range of 10³ to 10⁻¹⁵ may be reasonable for higher order coefficients.

The controller 146 may be configured to display the calculated flow rate on the display 158 of the user interface 148, as shown at block 306. The controller 146 may be configured to automatically calculate and display the flow rate (e.g., without requiring user intervention) in real time. In some cases, the controller 146 may calculate and display the flow rate continuously or at pre-defined intervals. It is contemplated that the controller 146 may be determining the flow rate and the left ventricle pressure substantially simultaneously. However, this is not required.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A percutaneous circulatory support device, comprising:
an impeller;
a motor configured to rotate the impeller to cause blood flow through the percutaneous circulatory support device;
a pressure sensor configured to measure a vascular pressure; and
a controller operably coupled to the motor and the pressure sensor, the controller configured to:
determine a working voltage applied to the motor;
determine a working speed of the motor; and
calculate at least one of:
i) a transvalvular pressure based on the working voltage and the working speed; or
ii) a blood flow rate through the percutaneous circulatory support device based on the working voltage and the working speed.

2. The device of claim 1, wherein the controller is configured to determine a left ventricular pressure based on the measured vascular pressure and calculated transvalvular pressure.

3. The device of claim 2, wherein the controller is configured to calculate the transvalvular pressure using a polynomial model of the working voltage and the working speed.

4. The device of claim 3, wherein the polynomial model is a fifth-order polynomial model.

5. The device of claim 1, wherein the controller is configured to perform auto-time alignment between pressure measurements and motor parameters.

6. The device of claim 5, wherein the controller is configured to calculate the blood flow rate using a polynomial model of the working voltage and the working speed.

7. The device of claim 6, wherein the polynomial model is a fifth-order polynomial model.

8. The device of claim 1, wherein the controller is configured to display the determined left ventricular pressure and/or blood flow rate on a display screen of the percutaneous circulatory support device.

9. The device of claim 1, wherein the controller is configured to determine the working voltage of the motor independent of the working speed of the motor.

10. A system for determining cardiac parameters, comprising:
a percutaneous blood pump including an impeller and a motor;
a pressure sensor;
a display; and
a controller operably coupled to the motor, the pressure sensor, and the display, the controller configured to:
determine a working voltage applied to the motor;
determine a working speed of the motor independent of the working voltage; and
calculate at least one of:
i) a transvalvular pressure based using a first polynomial model of the working voltage and the working speed; or
ii) a blood flow rate through the percutaneous circulatory support device based using a second polynomial model of the working voltage and the working speed.

11. The system of claim 10, wherein the controller is configured to determine a left ventricular pressure based on the measured vascular pressure and the calculated transvalvular pressure.

12. The system of claim 11, wherein the first polynomial model is a fifth-order polynomial with cross-terms between the working voltage and the working speed.

13. The system of claim 11, wherein the controller is configured to display the determined left ventricular pressure and/or the blood flow rate on the display.

14. The system of claim 10, wherein the second polynomial model is a fifth-order polynomial with cross-terms between the working voltage and the working speed.

15. The system of claim 10, wherein the working speed is measured based on a frequency of voltage switching signals generated as the motor rotates.

16. A method of operating a percutaneous circulatory support device, comprising:
positioning a blood pump within a patient's vasculature, the blood pump operably coupled to a controller;
collecting, at the controller, a working voltage of a motor operating the blood pump;
collecting, at the controller, a working speed of the motor;
measuring an aortic pressure; and
calculating, at the controller, at least one of:
i) a transvalvular pressure based on the working voltage and the working speed; or
ii) a blood flow rate through the percutaneous circulatory support device based on the working voltage and the working speed.

17. The method of claim 16, further comprising determining, at the controller, a left ventricular pressure based on the measured aortic pressure and the calculated transvalvular pressure.

18. The method of claim 16, wherein calculating the transvalvular pressure includes using a first polynomial model of the working voltage and the working speed and/or calculating the blood flow rate includes using a second polynomial model of the working voltage and the working speed.

19. The method of claim 18, wherein the first polynomial model is a fifth-order polynomial model and/or the second polynomial model is a fifth-order polynomial model.

20. The method of claim 16, further comprising displaying the determined left ventricular pressure and/or the blood flow rate on a display.
